(19) United States
(12) Patent Application Publication
(10) Pub. No.: US 2010/0124576 A1
(43) Pub. Date: May 20, 2010

(54) Topical Treatment Formulation of Natural Ingredients for Enhancing Sexual Response

(76) Inventors: Teri Amato, Bayside, NY (US); Chandrakala Agarwala, Las Vegas, NV (US)

Correspondence Address: HOFFMANN & BARON, LLP, 6900 JERICHO TURNPIKE, SYOSSET, NY, 11791, US

(21) Appl. No.: 12/271,258

(22) Filed: Nov. 14, 2008

(51) Int. Cl. A61K 31/195; A61K 36/886; A61P 15/00

(52) U.S. Cl. 424/744; 514/565

(57) ABSTRACT

The present invention relates to a topical composition to enhance sexual response. The composition includes L-arginine, trans-resveratrol, and a topical carrier wherein L-arginine is present in an amount not greater than about 10% and trans-resveratrol is present in an amount not greater than about 10% based upon the weight of the total composition. The invention also relates to a method of enhancing sexual response by topically applying the topical composition to the genitalia of a human, and an integrated product including the topical composition, a container for instantaneous delivery, and instructions for use.

### BACKGROUND OF THE INVENTION

The present invention relates to enhancing the human sexual experience, and, in particular, to a composition and use thereof to help stimulate sexual response.

Human sexual response involves many factors including psychological, hormonal, and physiological influences. The factors that affect sexual response can be named, but how they interact to product a sexual response is not fully understood.

For example, the female response has been divided into four phases:
1) Desire—including fantasies about sexual activity and the desire to have sexual activity;
2) Excitement—consisting of subjective senses of sexual pleasure and accompanying physiological changes including vasocongestion in the pelvis, vaginal lubrication, and expansion and swelling of the external genitalia;
3) Orgasm—consisting of peaking of sexual pleasure with release of sexual tension; and
4) Resolution—consisting of a sense of muscular relaxation and general well-being.
   (Adapted from Diagnostic and Statistical Manual IV, “Sexual and Gender Identity Disorder,” American Psychiatric Association, Washington, D.C., pp. 493-494 and 735-751, 1994).

In the case of male sexual performance, the ability to cause and sustain an erection is a complex phenomenon generally caused by an action of the automatic nervous system. The automatic nervous system controls blood flow in the penis by means of peripheral nerves attached to arterial vessels in and around the corpora cavernosa. Sympathetic nerves maintain these arteries in a constricted state. When aroused, the male parasympathetic system releases principally catecholamines which inhibit the sympathetic nerves resulting in relaxation of the smooth muscles surrounding the arteries and the dilation thereof. This results in an erection.

In both cases, arousal, response, and performance are part of a complicated cascade of events deriving from the psychological and physical characteristics of each partner. Consequently, it would be beneficial to be able to enhance sexual performance with an agent which improves the physical aspect while having the lowest psychological and emotional impact. For many people, a natural product would be ideal for enhancing sexual activity/performance as long as it does not have any inherent drawbacks.

Due to the complex nature of sexual response, there are few natural products capable of enhancing the sexual experience in both women and men. Many, if not most, natural products are unappealing to the senses, e.g., touch and smell, and may not be able to be used with physical forms of contraception such as condoms and spermicidal jellies.

Thus, there remains a need for compositions for enhancing sexual response in males and females that contain natural ingredients that do not interfere with the effectiveness of condoms. There is also a need for a method of enhancing sexual response and an integrated product for delivery of a composition for enhancing sexual response.

### SUMMARY OF THE INVENTION

The present invention relates to a topical composition including L-arginine, trans-resveratrol, and a topical carrier. The L-arginine is present in an amount not greater than about 10% and the trans-resveratrol is present in an amount not greater than about 10% based upon the weight of the total composition.

In a preferred embodiment, the topical carrier is a gel, cream, ointment, or lotion. In a most preferred embodiment, the topical carrier is a gel.

L-arginine is preferably present in an amount not less than about 1% and more preferably in an amount not less than about 2%. L-arginine is preferably present in an amount not greater than about 5%, more preferably not greater than about 4%, and most preferably not greater than about 3%.

Trans-resveratrol is preferably present in an amount not less than about 1% and more preferably in an amount not less than about 2%. Trans-resveratrol is preferably present in an amount not greater than about 5%, more preferably not greater than about 4%, and most preferably not greater than about 3%.

The topical composition may further comprise aloe vera gel powder 200X.

In another aspect, the invention relates to a method of enhancing sexual response by topically administering to the genitalia of a human, a topical composition including L-arginine, trans-resveratrol, and a topical carrier. The topical composition is as described above. In one embodiment, the human genitalia is a penis. In another embodiment, the human genitalia is the clitoris, labia, outer area of the vagina, or combinations thereof.

In yet another aspect, the invention relates to an integrated product including a topical composition, a container for instantaneous delivery, and instructions for use. The topical composition is as described above. In one embodiment, the container for instantaneous delivery is a tube or a pressurized delivery container.

The topical composition, method of enhancing sexual response, and integrated product are specifically directed to improving the sexual experience. The topical composition applied prior to sexual intercourse is useful in enhancing sensitivity of human genitalia to stimulation. The topical composition is preferably applied at least about 5 minutes prior to sexual intercourse, and more preferably at least about 10 minutes prior to sexual intercourse.

For a better understanding of the present invention, together with other and further advantages, reference is made to the following detailed description, and its scope will be pointed out in the claims.

### DETAILED DESCRIPTION

The topical compositions of the invention include L-arginine, trans-resveratrol, and a topical carrier.

L-arginine is a basic amino acid. Its structure is shown below.

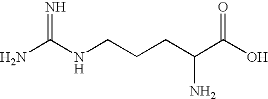

The topical compositions of the invention contain L-arginine in an amount to be effective in enhancing sexual response. Typically L-arginine not greater than about 10% by weight. In other embodiments, the topical compositions contain a maximum amount of L-arginine of about 5% by weight, more preferably about 4% by weight, and most preferably about 3% by weight. The topical compositions may contain a minimum amount of L-arginine of about 1% and most preferably about 2% by weight.

Trans-resveratrol is a naturally occurring phytoalexin than can be derived from muscadine grapes or produced synthetically. The structure of trans-resveratrol is shown below.

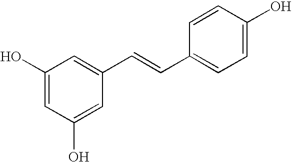

The topical compositions of the invention contain trans-resveratrol in an amount more greater than about 10%. In other embodiments, the topical compositions contain a maximum amount of trans-resveratrol of about 5% by weight, more preferably about 4% by weight, and most preferably about 3% by weight. The topical compositions may contain a minimum amount of trans-resveratrol of about 1% and most preferably about 2% by weight.

### Topical Carriers

The topical compositions also contain a topical carrier. The topical carriers useful for the delivery of the compounds of the invention can be any carrier known in the art for topical administration to the skin, for example, but not limited to, solvents such as a polyalcohol or water; emulsions (either oil-in-water or water-in-oil emulsions), such as creams or lotions; micro emulsions; gels; ointments; and aqueous solutions or suspensions.

### Gels, Emulsions, and Ointments as Topical Carriers

In a preferred embodiment, the topical carrier used to deliver the compounds of the invention is a gel, emulsion, or ointment. The most preferred topical carrier is a gel.

Gels are semisolid systems that contain suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention may be two-phase or single-phase systems. Some examples of suitable gels are disclosed in REMINGTON: THE SCIENCE AND PRACTICE OF PHARMACY 1517-1518 (Alfonso R. Gennaro ed. 19th ed. 1995), wherein the description of gels is incorporated herein by reference.

Gelling agents, that may be used include those known to one skilled in the art, such as hydrophilic and hydroalcoholic gelling agents frequently used in the cosmetic and pharmaceutical industries. The preferred gelling agent is aloe vera gel powder 200X.

In other embodiments, the hydrophilic or hydroalcoholic gelling agent may comprise “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

“CARBOPOL®” is one of numerous cross-linked acrylic acid polymers that are given the general adopted name carbomer. “Carbomer” is the USP designation for various polymeric acids that are dispersible but insoluble in water. When the acid dispersion is neutralized with a base a clear, stable gel is formed. The preferred carbomer is Carbomer 934P because it is physiologically inert and is not a primary irritant or sensitizer. Other carbomers include 910, 940, 941, and 1342.

Carbomers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling agents include hydroxyethylcellulose, cellulose gum, MVE/MA decadiene crosspolymer, PVM/MA copolymer, or a combination thereof.

Emulsions, such as creams and lotions are suitable topical formulations for use in the invention. An emulsion is a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In another embodiment, the topical carrier used to deliver the compounds of the invention is an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995), hereby incorporated herein by reference.

### Excipients

The compositions of the invention may include cosmetic or pharmaceutically acceptable excipients including, but not limited to, protective agents, adsorbents, demulcents, emollients, preservatives, anti-oxidants, moisturizers, buffering agents, solubilizing agents, skin-penetration agents, and surfactants. Pharmaceutically acceptable excipients are listed in REMINGTON: THE SCIENCE AND PRACTICE OF PHARMACY 866-885(Alfonso R. Gennaro ed. 19th ed. 1995; Ghosh, T. K.; et al. TRANSDERMAL AND TOPICAL DRUG DELIVERY SYSTEMS (1997), wherein the description of excipients is incorporated herein by reference.

Suitable protective agents and/or cosmetic agents, and adsorbents include, but are not limited to, dusting powders, zinc sterate, collodion, dimethicone, silicones, zinc carbonate, aloe vera gel and other aloe products, vitamin E oil, allatoin, petrolatum, titanium dioxide, and zinc oxide. The preferred protective agent is aloe vera gel.

Suitable demulcents include, but are not limited to, benzoin, hydroxypropyl cellulose, hydroxypropyl methylcellulose, hydroxyethyl cellulose, and polyvinyl alcohol. The preferred demulcent is hydroxyethyl cellulose.

Suitable emollients include, but are not limited to, animal and vegetable fats and oils, myristyl alcohol, alum, glycereth-26, and aluminum acetate. The preferred emollient is glycereth-26.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; parabens such as methylparaben, ethylparaben, propylparaben, and butylparaben; antibacterial esters, for example, esters of parahydroxybenzoic acid; and other anti-microbial agents such as chlorhexidine, chlorocresol, benzoic acid, polymyxin, and phenoxyethanol. The preferred preservatives are methylparaben and phenoxyethanol.

In a preferred embodiment, the minimum amount of preservative in the composition is about 0.1% and most preferably, about 0.2%.

In another preferred embodiment, the maximum amount of preservative in the composition is about 1%, more preferably about 0.75%, and most preferably about 0.5%.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol. The preferred moisturizer is propylene glycol.

In a preferred embodiment, the minimum amount of moisturizer in the composition is about 0.1%, more preferably, about 0.2%, and most preferably about 0.3%.

In another preferred embodiment, the maximum amount of moisturizer in the composition is about 1%, more preferably about 0.8%, and most preferably about 0.6%.

Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone. The preferred skin-penetrating agent is propylene glycol.

### Methods of Use

The topical compositions of the invention may be applied topically to human genitalia to enhance sexual response. The composition was formulated to increase sexual sensation, pleasure, and satisfaction.

The topical compositions are preferably applied about 5 minutes prior to engaging in sexual intercourse, and in another embodiment about 10 minutes prior to engaging in sexual intercourse. The topical compositions may be safely reapplied to the skin for the desired effect.

### Integrated Product

Another embodiment of the invention is an integrated product including the topical composition described above, a container for instantaneous delivery, and instructions for use. The container may be any container that can provide instantaneous delivery of the topical compositions. Preferred containers are tubes, squirt bottles, or pressurized delivery containers. The most preferred container is a polyethylene tube.

The present invention may be better understood by reference to the following examples. The following examples illustrate the present invention and are not intended to limit the invention or its scope in any manner.

### EXAMPLES

### Example I

### Gel Formulation for Male Use

| Ingredient | Weight Percent |
|---|---|
| DI Water | 47% |
| Aloe Vera Gel Powder 200X | 40% |
| L-Arginine | 2.5% |
| Glycereth-26 | 2.5% |
| Hydroxyethyl Cellulose | 2% |
| Trans-resveratrol | 2% |
| Propylene Glycol | 0.5% |
| Diazolidinyl Urea | 0.25% |
| Methylparaben | 0.25% |
| Menthol | 1% |
| TOTAL | 100% |

### Example II

### Gel Formulation for Female Use

| Ingredient | Weight Percent |
|---|---|
| DI Water | 50% |
| Aloe Vera Gel Powder 200X | 40% |
| L-Arginine | 2.5% |
| Glycereth-26 | 2% |
| Hydroxyethyl Cellulose | 2% |
| Trans-resveratrol | 2% |
| Propylene Glycol | 0.5% |
| Diazolidinyl Urea | 0.25% |
| Methylparaben | 0.25% |
| Menthol | 0.5% |
| TOTAL | 100% |

Thus, while there have been described what are presently believed to be the preferred embodiments of the present invention, those skilled in the art will appreciate other and further changes and modifications thereto, and it is intended to include such other changes as come with the scope of the invention as set forth in the following claims.

We claim:

1. A topical composition comprising:
L-arginine, trans-resveratrol, and a topical carrier wherein;
i) the L-arginine is present in an amount not greater than about 10%; and
ii) the trans-resveratrol is present in an amount not greater than about 10%.

2. A topical composition according to claim 1, wherein the topical carrier is a gel, cream, ointment, or lotion.

3. A topical composition according to claim 1, wherein L-arginine is present in an amount not less than about 1%.

4. A topical composition according to claim 1, wherein L-arginine is present in an amount not less than about 2%.

5. A topical composition according to claim 1, wherein L-arginine is present in an amount not greater than about 5%.

6. A topical composition according to claim 1, wherein L-arginine is present in an amount not greater than about 4%.

7. A topical composition according to claim 1, wherein L-arginine is present in an amount not greater than about 3%.

8. A topical composition according to claim 1, wherein trans-resveratrol is present in an amount not less than about 1%.

9. A topical composition according to claim 1, wherein trans-resveratrol is present in an amount not less than about 2%.

10. A topical composition according to claim 1, wherein trans-resveratrol is present in an amount not greater than about 5%.

11. A topical composition according to claim 1, wherein trans-resveratrol is present in an amount not greater than about 4%.

12. A topical composition according to claim 1, wherein trans-resveratrol is present in an amount not greater than about 3%.

13. A topical composition according to claim 1, further comprising aloe vera gel powder 200X.

14. A method of enhancing sexual response, comprising:
topically administering to the genitalia of a human a topical composition comprising
L-arginine, trans-resveratrol, and a topical carrier.

15. The method according to claim 14, wherein the topical compositions is applied at least about 5 minutes prior to sexual intercourse.

16. The method according to claim 14, wherein the topical compositions is applied at least about 10 minutes prior to sexual intercourse.

17. The method according to claim 14, wherein:
i) the L-arginine is present in an amount not greater than about 10%; and
ii) the trans-resveratrol is present in an amount not greater than about 10%.

18. The method according to claim 14, wherein the topical carrier is a gel, cream, ointment, or lotion.

19. The method according to claim 14, wherein L-arginine is present in an amount not less than about 1%.

20. The method according to claim 14, wherein L-arginine is present in an amount not less than about 2%.

21. The method according to claim 14, wherein L-arginine is present in an amount not greater than about 5%.

22. The method according to claim 14, wherein L-arginine is present in an amount not greater than about 4%.

23. The method according to claim 14, wherein L-arginine is present in an amount not greater than about 3%.

24. The method according to claim 14, wherein trans-resveratrol is present in an amount not less than about 1%.

25. The method according to claim 14, wherein trans-resveratrol is present in an amount not less than about 2%.

26. The method according to claim 14, wherein trans-resveratrol is present in an amount not greater than about 5%.

27. The method according to claim 14, wherein trans-resveratrol is present in an amount not greater than about 4%.

28. The method according to claim 14, wherein trans-resveratrol is present in an amount not greater than about 3%.

29. The method according to claim 14, wherein the composition further comprises aloe vera gel powder 200X.

30. The method according to claim 14, wherein the genitalia is a penis.

31. The method according to claim 14, wherein the genitalia is clitoris, labia, outer area of vagina, or combinations thereof.

32. An integrated product comprising:
a topical composition, a container for instantaneous delivery, and instructions for use, wherein the topical composition comprises:
L-arginine, trans-resveratrol, and a topical carrier wherein;
i) the L-arginine is present in an amount not greater than about 10%; and
ii) the trans-resveratrol is present in an amount not greater than about 10%.

33. An integrated product according to claim 32, wherein the container for instantaneous delivery is a tube or a pressurized delivery container.

34. An integrated product according to claim 32, wherein the topical carrier is a gel, cream, ointment, or lotion.

35. An integrated product according to claim 32, wherein L-arginine is present in an amount not less than about 1%.

36. An integrated product according to claim 32, wherein L-arginine is present in an amount not less than about 2%.

37. An integrated product according to claim 32, wherein L-arginine is present in an amount not greater than about 5%.

38. An integrated product according to claim 32, wherein L-arginine is present in an amount not greater than about 4%.

39. An integrated product according to claim 32, wherein L-arginine is present in an amount not greater than about 3%.

40. An integrated product according to claim 32, wherein trans-resveratrol is present in an amount not less than about 1%.

41. An integrated product according to claim 32, wherein trans-resveratrol is present in an amount not less than about 2%.

42. An integrated product according to claim 32, wherein trans-resveratrol is present in an amount not greater than about 5%.

43. An integrated product according to claim 32, wherein trans-resveratrol is present in an amount not greater than about 4%.

44. An integrated product according to claim 32, wherein trans-resveratrol is present in an amount not greater than about 3%.
